(19) United States
(12) Patent Application Publication
(10) Pub. No.: US 2005/0048486 A1
(43) Pub. Date: Mar. 3, 2005

(54) Method of inactivating ribonucleases at high temperature

(76) Inventors: Fen Huang, Madison, WI (US); Christine Andrews, Cottage Grove, WI (US); John Shultz, Verona, WI (US)

Correspondence Address: DEWITT ROSS & STEVENS S.C.;US Bank Building, Suite 401, 8000 Excelsior Drive, Madison, WI, 53717-1914, US

(21) Appl. No.: 10/403,395

(22) Filed: Mar. 31, 2003

(51) Int. Cl. C12Q 1/68; C12N 9/99

(52) U.S. Cl. 435/6; 435/184

(57) ABSTRACT

Methods for protecting RNA from RNase degradation and for inactivating RNases in solution are disclosed. The invention includes methods for protecting RNA during storage, for performing quantitative PCR reactions, or for preparing cDNA. The method includes using a combination of an RNase inhibitor and high heat to render RNases inactive, even after the RNase inhibitor has been denatured by heating.

### FIELD OF THE INVENTION

The present invention is directed to methods for protecting ribonucleic acids (RNA) from degradation by ribonucleases (RNases). Specifically, the invention includes methods for protecting RNA from RNases during storage of the RNA, as well as methods for protecting RNA from RNases present in reagents used in scientific protocols that utilize RNA (such as reverse transcriptase-polymerase chain reactions, RT-PCR). The invention further includes methods to increase the sensitivity of RT-PCR.

### DESCRIPTION OF PRIOR ART

Ribonucleic acid (RNA) is an extremely important component of most biological systems. Its biologic roles include messenger RNA (mRNA), which carries the genetic code from the nucleus; ribosomal RNA (rRNA), which helps to translate the nucleic acid message to a polypeptide; and transfer RNA (tRNA), which functions to help decode messenger RNA. Further, RNAs are beginning to be recognized for a host of other regulatory functions, such as small interfering RNA and regulatory ribozymes, which have an enzymatic function. In some viruses, RNA carries the core genetic message itself.

Because of its importance in biological actions, RNA production and degradation are heavily regulated in vivo. While DNA is quite stable, an effect of its being a double-stranded molecule, RNA (a single-stranded molecule) is extremely susceptible to enzymatic degradation. Enzymatic degradation is carried out by a ubiquitous class of enzymes called ribonucleases (RNases).

RNases are extremely robust enzymes. Unlike most proteins, RNases are very difficult to degrade either by extreme pH or high temperature. There are several theories as to why RNases evolved to be so robust. They include protection from the consequences of translating degenerate RNA into proteins and regulation of intracellular RNA. In addition, although RNases can be temporarily denatured by high temperatures, some RNases renature upon cooling (a phenomenon called reversible thermal denaturation) so that denaturing RNases via high temperature alone is not an effective method for protecting RNA from RNases at, say, room temperature.

RNA is an extremely important tool in molecular biology. Due to the presence of introns in eukaryotic genomic DNA, the genetic message carried in genomic DNA is not directly translatable into proteins. Therefore, genomic DNA is a second choice when making libraries, cloning, and introducing genes into a cell on a plasmid or vector. The most desirable source for libraries is complementary DNA (cDNA). cDNA is made directly from mRNA which has been back-transcribed into DNA. This process requires isolation of mRNA which has gone through the process of intron removal, a process commonly referred to as “splicing.” During splicing, the non-translated introns are removed before the RNA is translated into protein. By using reverse transcriptase in the presence of nucleotide bases (including thymine, instead of the uracil found in RNA), a single-stranded DNA, complementary to the mRNA, can be synthesized.

Further replication of the single-stranded DNA transcript using DNA polymerase produces a double-stranded cDNA molecule having the sequence of the mRNA template. In addition, cDNA, like genomic DNA, is very stable; thus, its utility for molecular biological manipulations is magnified. The cDNA can be used for a variety of purposes, including amplification using PCR and the creation of cDNA libraries for use in cloning. By synthesizing cDNA, scientists have been able to create synthetic genes which, when transfected into an organism, can be directly translated into a functional protein. This capability would be impossible using the genomic DNA of a eukaryote because of the presence of introns. The introns must be properly spliced from the genomic sequence in order for a proper protein to result.

The synthesis of cDNA is not the only experimental use for RNA. Other uses, such as RNA vectors (see, for example, Zhang et al. (1997) Virology 233:327-338) and RNA probes, are also adversely affected by RNases. Therefore, one important research effort of the last few years has been the development of methods to protect RNA from RNases. In short, because the need to preserve RNA for analysis has been known for some time, a number of different approaches have been used for inhibiting RNAse activity. The RNase activity to be eliminated from the sample may be present either through co-purification of the RNase with the RNA, or may have been introduced into the sample from reagents used in processing the sample.

Several methods for inhibiting RNase activity have been developed. These methods include the use of diethylpyrocarbonate (DEPC), the use of RNase inhibitor proteins, and the use of ribose compounds that preferentially bind to the RNase.

One method of inhibiting RNase activity involves using the chemical agent diethylpyrocarbonate (DEPC). DEPC reacts with RNAses to inactivate the enzyme. However, the use of this type of chemical entity is not always convenient or even possible. (For example, due to adverse chemical reactions, solutions of Tris and MOPS cannot be treated with DEPC.) DEPC reacts with a number of different residues in RNases, leading to deactivation of the RNase enzyme. For example, in RNase A (EC 3.2.27.5), two histidine residues (His-12 and His-119) are key to the catalytic activity of the enzyme. DEPC reacts with the His-12 residue of RNase A to yield a carbamate-type bond, thus making this residue unavailable for reaction with RNA. (See Findlay et al. (1961) Nature 190:781-784; and Raines (1998) Chem Rev. 98:1045-1066.). In other types of RNases, DEPC interferes the ε-amino groups of lysine and the carboxylic groups of aspartate and glutamate, both intra- and inter-molecularly, to deactivate RNases. While treatment with DEPC is effective, its use is very laborious. DEPC is also a suspected carcinogen.

When using DEPC as protection against RNases, reagents, glassware, electrophoresis equipment, and any other labware that may come in contact with the RNA is rinsed in DEPC-treated water, then incubated at 37° C. for several hours to promote RNase degradation. The treated equipment is then autoclaved for approximately 30 minutes to destroy the DEPC. In addition, RNA solutions are stored in DEPC-treated water to protect the RNA during storage. When this method of storing RNA is used, the DEPC needs to be removed from the solution before using the RNA.

RNase inhibitor proteins were first identified as a protein that inhibited pancreatic RNase. This family of RNase inhibitor proteins was identified and purified from placental extracts. (See Blackburn, P. et al. (1977) J. Biol Chem. 252:5904-5910.) A gene for an RNase inhibitor was subsequently cloned from the placenta, and a recombinant RNase inhibitor protein developed. (See, for example, U.S. Pat. No. 5,552,302, to Lewis et al.) These inhibitor proteins function mechanistically by forming a very strong 1:1 complex between the inhibitor and the RNase.

The genes encoding the human placental inhibitor, as well as those from pig and rat, have been cloned and sequenced. The three-dimensional structures for some of the members of the family have also been determined. (See Kobe & Deisenhofer (1996) “Mechanism of ribonuclease inhibition by ribonuclease inhibitor protein based on the crystal structure of its complex with ribonuclease A,” J. Mol. Biol. 264(5):1028-1043.) Comparisons of the properties of this family of RNase inhibitor proteins have been published. (See Blackburn et al. (1977) J. Biol. Chem. 252:5904-5910; Burton & Fucci (1982) Int. J Pept. Protein Res. 19:372-379.) The usefulness of these inhibitor proteins in molecularbiology applications has resulted in their characterization to some extent. In particular, the human placental form of the inhibitor protein has been reported: (1) to inhibit RNases of the RNase A, B and C family of enzymes; (2) to be thermally inactivated at about 55° C. in aqueous solution; and (3) to be unable to inhibit the major RNase from E. coli (commonly referred to as RNAse 1) or RNases from plant sources. (See, for example, “Expressions 9.3,” a publication of Invitrogen Life Technologies (San Diego, Calif.) that describes Invitrogen's RNaseOUT-brand inhibitor. See also Ambion, Inc.'s (Austin, Tex.) product literature for Ambion's RNase Inhibitor.) When the RNAse is complexed to the inhibitor, the complex does not have any RNAse activity. However, as reported in the above-noted product literature, the RNAse is not permanently inactivated by the inhibitor. If the inhibitor is released from the inhibitor-RNase complex, under certain conditions the freed RNAse will regain its ability to degrade RNA.

The RNase inhibitor protein from human placenta-either isolated from its native source or made through recombinant means—has been available commercially for a number of years. During that time, reports have been published that the inhibitor is ineffective in preventing RNA degradation in certain molecular biology applications, such as RT-PCR. This is due, reportedly, to the poor thermostability of the inhibitor protein at the temperatures used in such reactions. In fact, these publications suggest that adding the RNase inhibitor would be detrimental to successful completion of RT-PCR experiments. In short, the product literature suggests that the RNase inhibitor protein as supplied may already have a significant fraction of the inhibitor protein complexed to RNase. Further, this RNAse would then be released in an active form upon heating of a solution containing the RNase inhibitor. The literature goes on to infer that the potentially active RNAse released may destroy the RNA template in the experiments, thus leading to failure in the experiments.

Due to the difficulty of protecting RNA from RNases, there is a long-felt and unmet need for a better method to protect RNA from RNase degradation, both during storage of the RNA and during manipulations of the RNA. The method should be easy to implement and should not require the use of toxic reagents. The method should yield RNase-protected RNA that can be directly used (from one protocol to the next) without intervening and additional purification steps and without concern for the enzymatic degradation of the RNA.

### SUMMARY OF THE INVENTION

The present inventors have discovered, quite surprisingly, that an RNase inhibitor protein from a mammalian source (human placenta, rat, etc., native or recombinant) can be combined with particular chemical reagents, such that the combination allows the inhibitor to be highly effective in specific, high-temperature applications, such as RT-PCR and quantitative RT-PCR. The combination of these materials, and in particular when combined with heating of the RNA inhibitor solution and reagents with a sample suspected of containing RNAse, results not only in the inhibition of RNAse in the reaction, but also results in the lack of release of active RNAse following treatment of the solution under conditions that inactivate the RNAse inhibitor. Insofar as the literature discussed previously directly indicates that RNAse inhibitor solutions should not be heated under any conditions (as they will inactivate the RNAse inhibitor and potentially release active RNAse into the experimental solution), the present invention is in direct conflict with the conventional fashion in which placental RNase inhibitor is used.

Another unexpected and unpredictable aspect of the present invention is that when the RNAse inhibitor solutions of the present invention are heated, the solutions are capable of inactivating RNAses not normally inhibited by the RNAse inhibitor alone or the added reagents alone. While not being limited to a specific mode of action, this increase in the range of RNAses capable of being inactivated apparently is the result of a synergism between the RNase inhibitor and the added reagents. The combination is greater than the sum of its parts; the combination inactivates RNases that are not inactivated by either the inhibitor or the added reagents separately. The net result is that the invention described and claimed herein results in the protection of RNA from mammalian RNAses both before and after heating of the solution, and also provides protection from RNAses derived from bacterial and plant sources after gently heating the solution.

It is therefore a primary aim and object of the invention to provide a method for protecting RNA from RNase degradation. A first embodiment of the invention is thus directed to a method for protecting RNA from enzymatic degradation by RNases. The method comprises first, to a first solution containing RNA or to which RNA will subsequently be added, adding an amount of a second solution comprising an amount of an RNase inhibitor protein and a buffer comprising 1,4-dithiothreitol DTT, to yield a mixture that comprises at least about 50 μM DTT. The amount of RNase inhibitor protein in the second solution is sufficient to protect RNA from enzymatic degradation by RNases present in the mixture. Then the mixture is heated to a temperature no less than about 70° C. for a time sufficient to inhibit RNase activity present in the mixture. In this fashion, RNA present in the mixture, or subsequently added to the mixture, is protected from enzymatic degradation by RNases in general, and mammalian RNases in particular. If RNA is to be subsequently added to the mixture, the mixture can be heated to at least about 90° C.

The preferred method protects RNA from enzymatic degradation by RNase A, RNase B, RNase C, and RNase 1.

The buffer containing the RNase inhibitor protein preferably contains sufficient DTT so that the final concentration of DTT in the mixture is at least about 50 μM DTT. The buffer can contain additional DTT as well. For example, the buffer can contain sufficient DTT so that the final concentration of DTT is the mixture is at least about 100 μM DTT, or even at least about 1.0 mM DTT.

The RNase inhibitor protein is preferably derived from porcine, rat, human placental, or recombinant human placental sources. Such RNases inhibitors are available commercially, such as from Promega Corporation.

To gain the benefits of the present invention, the mixture need not be heated for a long time. Generally, about one (1) minute at 70° C. or higher is sufficient. The mixture, of course, can be heated for much longer periods of time, anywhere from minutes (if RNA is present) to hours (if RNA is to be subsequently added).

A second embodiment of the invention is drawn to a method of inactivating RNases in a first solution known to contain RNA and suspected of containing RNases. This second embodiment comprises adding to the first solution a second solution comprising an RNase inhibitor protein deposited in a buffer comprising at least about 50 μM DTT, to yield a mixture, and then heating the mixture to a temperature of at least about 70° C. for a time sufficient to inhibit RNase activity present in the mixture. This results in RNases present in the first solution, if any, being inactivated. It is preferred that the solution be heated anywhere from one (1) minute to five (5) minutes.

A third embodiment of the invention is drawn to a method of storing RNA under conditions that protect the RNA from enzymatic degradation by RNases. The third embodiment comprising adding to a first solution containing isolated RNA or to which isolated RNA will subsequently be added, a second solution comprising an RNase inhibitor protein in a buffer comprising at least about 50 μM DTT, to yield a mixture. The mixture is then heated 70° C. for a time sufficient to inhibit RNase activity present in the mixture; and then the mixture is cooled and stored in an air-tight container.

Yet another embodiment of the invention is directed to a method of performing RT-PCR and quantitative RT-PCR. This fourth embodiment of the invention comprises first, prior to undergoing thermal cycling, adding to an RT-PCR reaction cocktail containing RNA (or to which RNA will subsequently be added) an amount of a solution comprising an RNase inhibitor protein in a buffer comprising at least about 50 μM DTT, to yield a mixture. The amount of the solution added is sufficient to protect any RNA present in the RT-PCR reaction cocktail from enzymatic degradation during a first round of thermocycling. Then, if RNA is absent from the mixture, adding RNA template to the mixture. An RT-PCR reaction is then conducted on the mixture, whereby RNA in the mixture is protected from enzymatic degradation by RNases present in the RT-PCR reaction cocktail and is also protected from enzymatic degradation by RNases during the first round of thermocycling and throughout the RT-PCR reaction.

A variation on this embodiment comprises adding a first solution containing an RNase inhibitor protein in a buffer to an RT-PCR reagent mixture, the buffer comprising at least about 50 μM DTT, to yield a second solution. The second solution is then heated to at least about 70° C. for a time sufficient to inhibit RNase activity present in the second solution. RNA is then added to the second solution to yield an RNA mixture. Lastly, an RT-PCR reaction is conducted on the RNA mixture, whereby the RNA in the RNA mixture is protected from enzymatic degradation by RNases present in the second solution and whereby the RNA in the mixture is further protected from RNases during the RT-PCR reaction.

A still further embodiment of the invention is directed to a method of inactivating RNase I. This embodiment of the invention comprises adding to a first solution suspected of containing RNase I, a second solution comprising an RNase inhibitor protein in a buffer comprising at least about 50 μM DTT, to yield a mixture; and then heating the mixture to a temperature of at least about 70° C. for a time sufficient to inhibit RNase I activity present in the mixture, whereby any RNase I present in the first solution is inactivated.

In any of the embodiments disclosed herein, the RNase inhibitor protein used in the method can be derived from porcine, rat, human placental or recombinant human placental sources.

The objects and advantages of the invention will appear more fully from the following detailed description of the preferred embodiment of the invention made in conjunction with the accompanying figures.

### BRIEF DESCRIPTION OF THE FIGURES

FIG. 1 is a photograph of a gel illustrating inhibition of bovine pancreatic RNase using rat-derived RNase inhibitor protein in an RT-PCR protocol. See Example 1 for lane assignments.

FIG. 2 is a photograph of a gel illustrating protection of mRNA in quantitative RT-PCR using rat-derived RNase inhibitor protein. See Example 2 for lane assignments.

FIG. 3 is a photograph of a gel illustrating protection of mRNA in quantitative RT-PCR using human-derived RNase inhibitor protein. See Example 2 for lane assignments.

FIG. 4 is a histogram showing the results of a statistical analysis of band density for the products of the RT-PCR reactions described Example 2 and shown in the gels of FIGS. 2 and 3.

FIG. 5 is a schematic showing of the results of a plate assay indicating the digestion of RNA by RNase. The assay comprises an agar plate loaded with agar mixed with RNA and a pH indicator. The plate is cored and the wells loaded with RNase and an RNase inhibitor, in treatments that are either heated or not. Digestion of RNA results in a visible digestion zone around the affected wells. See Example 3.

FIG. 6 shows the results of a plate assay to examine the effect of heating RNase on the degradation of RNA in the presence of an RNase inhibitor and different types of buffers. See Example 4.

FIG. 7 is a photograph of a gel illustrating protection of mRNA from degradation by RNase derived from wheat germ in an RT-PCR experiment. See Example 5.

FIG. 8 is a photograph of a gel illustrating protection of mRNA from degradation by RNase derived from wheat germ in an RT-PCR experiment. See Example 6.

### DETAILED DESCRIPTION OF THE INVENTION

The present invention is directed to methods for protecting RNA from degradation by RNases. The invention is further directed to methods of storing RNA in an RNase activity-free stock solution.

Abbreviations and Definitions:

As used herein, the term “RNA” expressly denotes RNA from any source without limitation, including prokaryotic RNA, eukaryotic RNA, mitochondrial RNA, and RNA derived from transcription reactions.

As used herein, the unqualified term “RNase” expressly denotes RNase from any source without limitation, including prokaryotic and eukaryotic RNases. RNases are found in most organisms and in many organs and body fluids. Examples of RNases include (without limitation) RNases A, B, and C (mammalian, e.g., bovine pancreatic), RNase 1 (e.g., human pancreatic), RNase 2 (eosinophil-derived neurotoxin), RNase 3 (eosinophil-cationic protein), RNase 4, and RNase 5, as well as the bacterial RNases I, II, III, P, PH, R, D, T, BN, E, and M, among others. All share the primary activity of degrading RNA. For a more extensive discussion of RNases, see, for example, D'Allesio & Riordan “Ribonucleases: Structures and Functions,” Academic Press, New York (1997); Sorrentino & Libonati (1997) “Structure-Function Relationships in Human Ribonucleases: Main Distinctive Features of the Major RNases,” FEBS Letters 404:1-5; and Nicholson (1999) “Function, Mechanism, and Regulation of Bacterial Ribonucleases,” FEMS Microbiol. Rev. 23:371-390.

As used herein, the terms “RNase inhibitor protein” or “RNase inhibitor” denotes a mammalian-derived protein that inhibits the activity of RNase. The preferred RNase inhibitor proteins for use in the present invention are those manufactured by Promega Corporation, Madison, Wis. Promega markets RNase inhibitor proteins derived from human placenta, both as a native protein and a recombinant version, under the trademark “RNasin”-brand RNase inhibitor. For additional information on the RNasin-brand RNase inhibitor, see Blackburn & Moore (1982) In: The Enzymes, Vol. XV, Part B; Blackburn, Wilson, & Moore, (1977) J. Biol. Chem. 252:5904; Lee et al. (1989) Biochemistry 28:219; Lee et al. (1989) Biochemistry 28, 225. See also U.S. Pat. Nos. 4,966,964; 5,019,556; and 5,266,687.

Another preferred RNase inhibitor protein for use in the present invention is designated herein as “RNasin-Plus” RNase inhibitor. This RNase inhibitor protein is a recombinant protein derived from rat lung and produced in E. coli. For a description of the cloning of this protein, see Kawanomoto et al. (1992), Biochim. Biophys. Acta 1129: 335-338, which discusses the cDNA cloning and sequence of rat ribonuclease inhibitor isolated from a rat lung cDNA library. This protein can be purchased commercially from Roche Diagnostics, a division of F. Hoffmann-La Roche Ltd., Basel, Switzerland. The cloned RNA encoding this rat-derived RNasin is also available commercially from OriGene Technologies, Inc. (Rockville, Md.).

The invention described herein is suitable for use in a variety of molecular biological protocols that use or require RNA. For an overview of a host of such protocols, see “RNA Methodologies, Second Edition,” E. Farrell, Jr., editor, Academic Press, 1998.

The following primers where used in the Examples:

Luciferase RT-PCR reverse primer

| | |
|---|---|
| F-CGCCCCCTCGGAG: | (SEQ. ID. NO: 1) |

Forward luc RT-PCR

| | |
|---|---|
| F-GAAAGGCCCGG: | (SEQ. ID. NO: 2) |

downstream Kan RT-PCR

| | |
|---|---|
| F-GGGATCCTCTAGAGTCGCCA: | (SEQ. ID. NO: 3) |

upstream Kan RT-PCR 2

| | |
|---|---|
| F-TTGGGCGTGTCTCAAAATCT: | (SEQ. ID. NO: 4) |

Luciferase RT-PCR reverse primer

| | |
|---|---|
| HO-CGCCCCCTCGGAG: | (SEQ. ID. NO: 5) |

Forward primer luc RT-PCR

| | |
|---|---|
| HO-GAAAGGCCCGG: | (SEQ. ID. NO: 6) |

In an exemplary version of the instant invention, a first solution containing RNA is protected against degradation by RNases by adding to it a second solution containing an RNase inhibitor, such as “RNasin-Plus” brand RNase inhibitor (Promega), in a buffer. The buffer includes at least about 50 μM DTT (most preferred) and may contain at least about 100 μM and even at least about 1.0 mM DTT. In a particularly preferred embodiment, the buffer comprises Promega Buffer B or Promega Storage buffer. Promega BufferB comprises 6 mM Tris-HCl (pH 7.5), 6 mM MgCl₂, 50 mM NaCl, and 1.0 mM DTT. Promega Storage Buffer comprises 20 mM HEPES-KOH (pH 7.6), 50 mM KCl, 50% (v/v) glycerol, and 8 mM DTT.

After adding the RNase inhibitor and buffer, the solution is heated to at least about 70° C. for a time sufficient to inactivate RNases, generally from about one (1) minute to perhaps five (5) minutes or more. The time the solution is left at elevated temperatures will, to some extent, depend upon the protocol being undertaken. Inactivation of the RNases occurs essentially immediately for mammalian RNases, and the heating serves to deactivate more hardy RNases. If RNA is not yet present in the mixture, it may be heated for 30 minutes or longer at temperatures at least as high as 90° C. This treatment renders the mixture free from RNase activity both before and after the heating step. A distinct advantage of this approach is that the RNA in the solution is protected from RNases for an extended period of time without fear of reversible denaturation. In short, once treated, the RNA remains protected from RNases so long as the container in which it is stored is not opened. (Opening the storage container potentially introduces non-heat treated RNases into the treated RNA solution.)

The RNase inhibitors that can be used in the invention include, without limitation, porcine RNase inhibitor, rat RNase inhibitor, human placental RNase inhibitor, and recombinant RNase inhibitor. This list is exemplary. There are several commercial suppliers of RNase inhibitor, including Promega Corporation.

Another embodiment of the invention is a method of protecting RNA from RNases during storage. In this embodiment, the solution containing RNA can be stored for long periods of time without concern for the degradation of the RNA by RNases. To protect the RNA, an RNase inhibitor, such as “RNasin” brand inhibitor, together with the appropriate buffer, is added to the RNA-containing solution. The solution is then heated to about at least 70° C. for about at least 1 minute. The mixture is then placed in a suitable container and allowed to cool. After being so treated, the RNA solution can be stored for extended periods of time (i.e., at least one (1) hour and often far longer) at room temperature, yet still be protected from RNases (so long as the container is not opened). A distinct advantage of this embodiment is that the treated RNA solution does not have to be placed in cold storage to be protected from RNases. However, those of skill in the art will understand that cold storage would help alleviate temperature-dependent RNA degradation.

The invention also comprises a method to protect RNA during chemical and enzymatic reactions in general and, in particular, during RT-PCR-based protocols. In this embodiment of the invention, the RNA may have been isolated previously and may have already been protected from RNases by the disclosed invention. However, those of skill in the art will recognize that adding any reagent to an RNA-containing solution risks the introduction of non-inhibited RNases. In such an instance, an RNase inhibitor can be added to the reaction mixture before the first reaction step is performed. In the case of RT-PCR-based reactions, the RNase inhibitor is added prior to the first thermocycling step. The RNA is thereby protected from degradation during the thermocycling step and, surprisingly, in all subsequent thermocycles. In a particularly preferred embodiment, the RNase inhibitor and buffer are added to the reaction mixture prior to the addition of the RNA. Further, the reaction mixture may be heated prior to addition of the RNA, assuring the highest RNA protection and the highest sensitivity of the reverse transcriptase reaction. In addition, as noted earlier, the contents of the reaction mixture continue to be protected from RNase degradation even after the PCR is performed, so long as additional RNases are not introduced to the reaction solution during subsequent manipulations.

The invention is also effective to inhibit RNases normally thought not to be inhibited by native mammalian or recombinant RNase inhibitors. A synergistic effect has been discovered in the combination of an RNase inhibitor protein, a buffer containing DTT, and heat, the combination yielding results that are greater than the sum of the individual steps alone. In this embodiment of the invention, RNase I which is produced by prokaryotes in general, and E. coli in particular, are inhibited. In this embodiment, as in the earlier embodiments, an RNase inhibitor and a suitable buffer are added to a solution thought to contain RNase I to yield a second solution. The second solution is then heated to at least about 70° C. for a time sufficient to inactivate the RNase I. The prokaryotic RNases are thus permanently inactivated by the treatment, and RNA can be added without fear of degradation.

While the above methods for inactivating RNases and protecting RNA from degradation can be effected by heating the RNA solution or mixture at 70° C., it is a feature of the invention that a solution or mixture to which RNA is to be subsequently added can be heated for an extended period of time at temperatures of at least as high as 90° C. or higher (essentially to the boiling point). Once the reaction is cooled, RNA can be added without fear of degradation by RNases. It is a further aspect of the invention that, upon addition of the RNase inhibitor protein to the RNA solution, the RNase will be inhibited from degrading the RNA in the solution. Moreover, after heating the mixture at a temperature of at least about 70° C., the RNases are inactivated and the RNA is safe from RNase degradation for an extended period of time (i.e., at least an hour or more), at room temperature.

### EXAMPLES

The following Examples are included solely to provide a more complete understanding of the invention disclosed and claimed herein. The Examples do not limit the invention in any fashion.

### Example 1

### Inactivation of RNase in Rat Liver Lysate by Promega's “RNasin-Plus”-Brand RNase Inhibitor

The purpose of this Example is to demonstrate the protection of mRNA with “RNasin Plus” RNase inhibitor in an RT-PCR experiment wherein rat liver lysate (a source of RNase) was purposefully added to the reactions.

Materials:

Rat Liver Lysate: 0.5 mg/ml in nanopure water (Sigma Pt #L-1380 Lt #108F8185)

Luciferase mRNA: 0.1 mg/ml in nanopure water (Promega Pt#L456A Lt #14937403)

Luciferase mRNA: 0.01 mg/ml in nanopure water (Promega Pt#L456A Lt #14937403)

“RNasin Plus”-brand RNase inhibitor*: 40 units/μl (Promega Pt #N261 Lt #165682)

AccessQuick™RT-PCR System (Promega Pt #A1703 Lt #158304)

* “RNasin Plus”-brand rat-derived RNase inhibitor can be purchased commercially from Promega.

Experimental:

Two hundred (200) μl of both the “RNasin Plus”-brand inhibitor and the rat liver lysate were heated in separate tubes for 15 minutes at 70° C.

The following reactions were then assembled in duplicate without the addition of mRNA.

| Rxn # | Nanopure Water | RNasin | Rat Liver Lysate |
|---|---|---|---|
| 1 | — | 20 μl | — |
| 2 | 17.5 μl | — | 2.5 μl |
| 3 | — | 20 μl | 2.5 μl |
| 4 | — | 20 μl | 2.5 μl |
| 5 | — | 20 μl | 2.5 μl |
| 6 | — | 20 μl | 2.5 μl |
| 7 | — | 20 μl | 2.5 μl |

| | | | Rat |
|---|---|---|---|
| Rxn # | Contents | RNasin Heated | Liver Lysate Heated |
| 1 | RNasin Only | No | NA |
| 2 | Rat Liver Lysate Only | NA | no |
| 3 | RNasin + Lysate | No | no |
| 4 | RNasin + Lysate | Yes | no |
| 5 | RNasin + Lysate | No | yes |
| 6 | RNasin + Lysate | Yes | yes |
| 7 | RNasin + Lysate | Yes | yes |

In Reaction Nos. 4, 5, and 6, the RNase inhibitor and the rat liver lysate were heated separately and then combined. In Reaction No. 7, the RNase inhibitor and the rat liver lysates were combined and then heated.

Reaction No. 7 was assembled using non-heat treated lysate and non-heat treated RNase inhibitor and then incubated at 70° C. for 15 minutes.

One (1) μl of 0.1 mg/ml luciferase mRNA (100 ng) was added to the first set of reactions.

One (1) μl of 0.01 mg/ml luciferase mRNA (10 ng) was added to the second set of reactions. That is, the second set of reactions included a 10-fold reduction in the amount of mRNA template as compared to the first set of reactions.

The reactions were then incubated for 1 hour at 37° C.

During the incubation, an RT-PCR master mix was assembled on ice using components available from Promega Corp., as follows:
250 μl Access Quick 2× Master Mix
220 μl Nuclease-free Water
10 μl Luciferase Upstream primer, Promega Part No.20247 (15 μM)
10 μl Luciferase Downstream primer Promega Part No.20818 (15 μM)
10 μl AMV-RT (5 units/μl)

After a 1 hour incubation at 37° C. (a temperature designed to challenge the reaction, 37° C. being an optimum temperature for RNase activity), the reactions were moved on ice. Forty-five (45) μl of the RT-PCR master mix was dispensed into “MicroAmp”-brand strip-well tubes on ice. Five (5) μl of each reaction was then added to the master mix. The reactions were placed in a PE 9600 thermocycler (PerkinElmer Corporation, Shelton, Conn.) and cycled as follows:

| | | |
|---|---|---|
| 48° C. | 45 minutes | 1 cycle |
| 96° C. | 2 minutes | 1 cycle |
| 94° C. | 15 seconds | |
| 65° C. -> 55° C. | 1 minute | 20 cycles |
| 72° C. | 1.5 minutes | |
| 72° C. | 5 minutes | 1 cycle |
| 4° C. | Soak | |

Fifteen (15) μl of each RT-PCR reaction was then loaded onto a 1% TBE agarose gel with ethidium bromide staining and run for 1 hour at 80V.

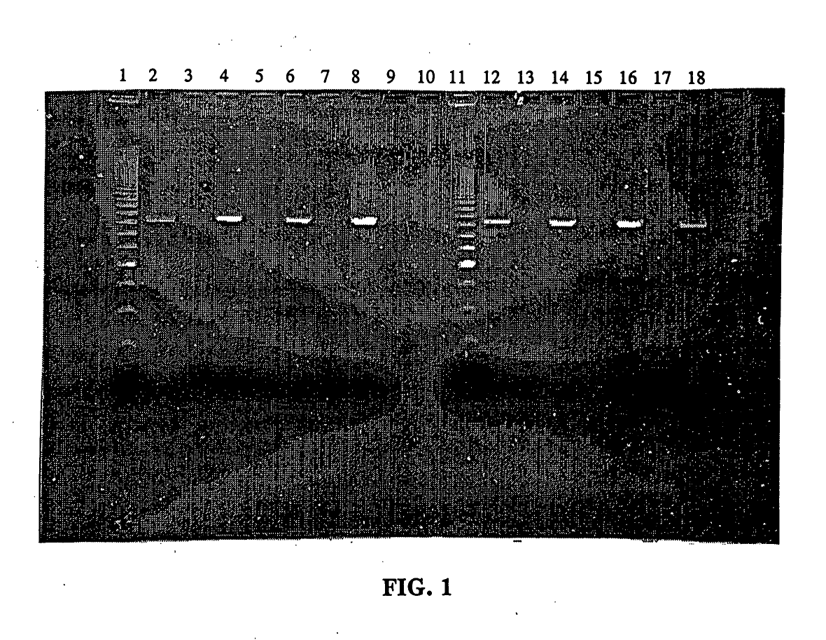

The results are shown in FIG. 1. The lane descriptions are as follows:
Lane Nos. 1 through 8 contain 100 ng mRNA.
Lane Nos. 11 through 18 contain 10 ng mRNA.
Lane Nos. 9 and 10 are blanks.
   Lane No.

1. 200 b.p. DNA Step Ladder

2. RNasin Plus Only (−)

3. Lysate Only (−)

4. RNasin (−)+Lysate (−)

5. RNasin (+)+Lysate (−)

6. RNasin (−)+Lysate (+)

7. RNasin (+)+lysate (+) heated separately

8. RNasin (+)+lysate (+) heated together

11. 200 b.p. DNA Step Ladder

12. RNasin Plus Only (−)

13. Lysate Only (−)

14. RNasin (−)+Lysate (−)

15. RNasin (+)+Lysate (−)

16. RNasin (−)+Lysate (+)

17. RNasin (+)+lysate (+) heated separately

18. RNasin (+)+lysate (+) heated together

(−)=Non heated sample

(+)=Heated sample

The results of the gel shown in FIG. 1 are striking. In each of lanes 3, 5, 7, 13, 15, and 17, there is a complete lack of RT-PCR product. In contrast, in each of lanes 2, 4, 6, 8, 12, 14, 16, and 18, there is a very distinct product detected. These results indicate a distinct synergy between the inhibitor, the RNase, and heating. In particular, as shown in lanes 7 and 17, when the inhibitor and the lysates are heated separately, and then combined, there is a total failure of RT-PCR. But, as evidenced by lanes 8 and 18, when the inhibitor and the lysates are combined and then heated, the RT-PCR experiment is a success. Not also that in lanes 5 and 15 (where the inhibitor is heated, but the lysate is not), and in lanes 7 and 17 (where the inhibitor and lysate are heated separately) the RT-PCR experiment fails (indicating that heating the inhibitor in the absence of the lysate “kills” the inhibitor). Surprisingly, however, when the inhibitor and the lysates are combined and then heated, as in lanes 8 and 18, the RT-PCR experiment is successful, indicating a synergy that is more than a sum of the separate effects of the inhibitor, the buffer solution, and heat.

### Example 2

### Protection of mRNA in Quantitative RT-PCR

The purpose of this Example is to demonstrate that the present invention will protect mRNA when rat-derived placental RNase inhibitor is used and when human-derived placental RNAse inhibitor is used in quantitative RT-PCR experiments wherein rat liver RNases are purposefully added to the reaction.

Materials:

Rat Liver Lysate: 0.5 mg/ml in nanopure water (Sigma Pt #L-1380, Lt #108F8185)

Luciferase mRNA: 0.1 mg/ml in nanopure water (Promega Pt#L456A, Lt #14937403)

Kanamycin mRNA: 0.005 mg/ml in nanopure water (Promega Pt #C138A, Lt #15423602)

“RNasin Plus”-brand inhibitor: 40 units/μl (Promega Pt #N261, Lt #165682)

Recombinant Rnasin Inhibitor: 40 units/∞l (Promega Pt #N25 1, Lt #152734)

AcessQuick™RT-PCR System (Promega Pt #A1703 Lt #158304)

Experimental:

The following reactions were assembled without the addition of mRNA:

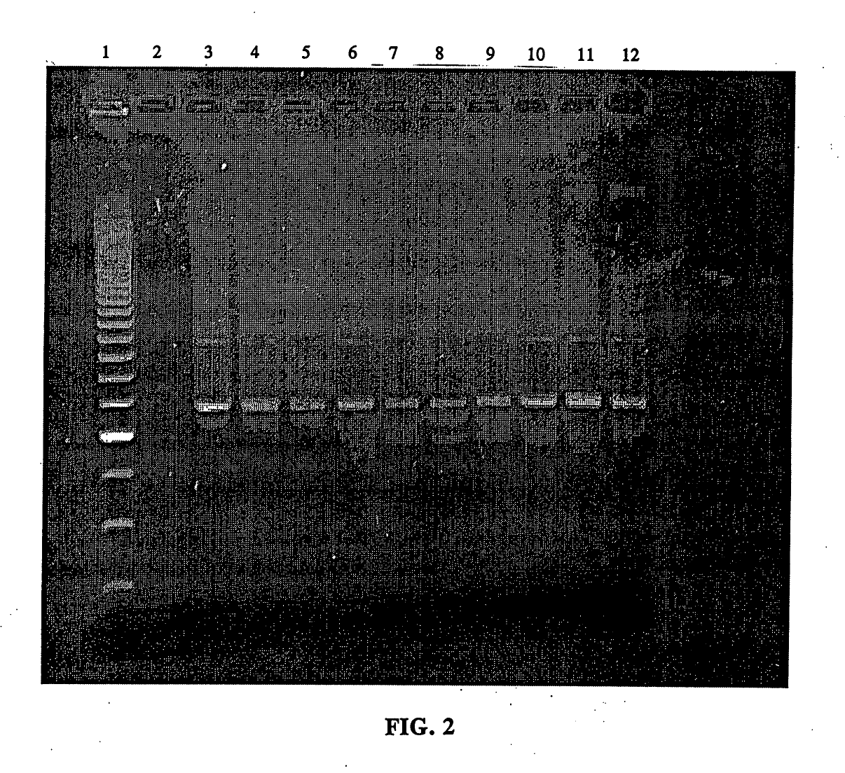

Rat Rnase Inhibitor (FIG. 2):

| Reaction | | | Rat Liver | Rat | |
|---|---|---|---|---|---|
| # | Luc RNA | Kan RNA | Lysate | RNasin | Nanopure |
| 1 | — | — | 2.5 μl | 20 μl | 4.5 μl |
| 2 | 2.5 μl | 2 μl | — | — | 23.0 μl |
| 3 | 2.5 μl | 2 μl | — | 20 μl | 2.5 μl |
| 4 | 2.5 μl | 2 μl | — | 20 μl | 2.5 μl |
| 5 | 2.5 μl | 2 μl | — | 20 μl | 2.5 μl |
| 6 | 2.5 ul | 2 ul | 2.5 ul | — | 20.0 ul |
| 7 | 2.5 ul | 2 ul | 2.5 ul | — | 20.0 ul |
| 8 | 2.5 ul | 2 ul | 2.5 ul | — | 20.0 ul |
| 9 | 2.5 ul | 2 ul | 2.5 ul | 20 ul | — |
| 10 | 2.5 ul | 2 ul | 2.5 ul | 20 ul | — |
| 11 | 2.5 ul | 2 ul | 2.5 ul | 20 ul | — |

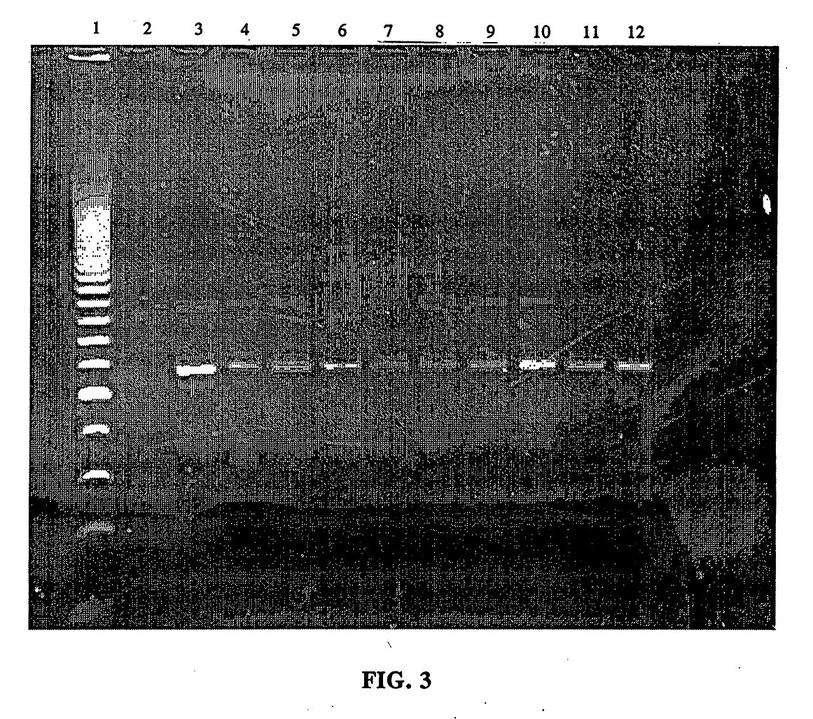

Human RNase Inhibitor (FIG. 3):

| Reaction | | | Rat Liver | Human | |
|---|---|---|---|---|---|
| # | Luc RNA | Kan RNA | Lysate | RNasin | Nanopure |
| 1 | — | — | 2.5 μl | 20 μl | 4.5 μl |
| 2 | 2.5 μl | 2 μl | — | — | 23.0 μl |
| 3 | 2.5 μl | 2 μl | — | 20 μl | 2.5 μl |
| 4 | 2.5 μl | 2 μl | — | 20 μl | 2.5 μl |
| 5 | 2.5 μl | 2 μl | — | 20 μl | 2.5 μl |
| 6 | 2.5 μl | 2 μl | 2.5 μl | — | 20.0 μl |
| 7 | 2.5 μl | 2 μl | 2.5 μl | — | 20.0 μl |
| 8 | 2.5 μl | 2 μl | 2.5 μl | — | 20.0 μl |
| 9 | 2.5 μl | 2 μl | 2.5 μl | 20 μl | — |
| 10 | 2.5 μl | 2 μl | 2.5 μl | 20 μl | — |
| 11 | 2.5 μl | 2 μl | 2.5 μl | 20 μl | — |

The reactions were incubated for 5 minutes at room temperature.

Luciferace mRNA, 2.5 μl of 0.1 mg/ml, (250 ng total) and 2 μl of 0.005 mg/ml kanamycin mRNA (10 ng) were then added to each reaction.

The reactions were incubated at 37° C. for 5 minutes.

An RT-PCR master mix was assembled on ice as follows, using components available from Promega Corp.:
250 μl Access Quick 2× Master Mix
200 μl Nuclease-Free Water
10 μl Luciferase Upstream primer #20939 (15 μM)
10 μl Luciferase Downstream primer #20979 (15 μM)
10 μl Kanamycin Upstream primer #20936 (15 μM)
10 μl Kanamycin Downstream primer #20937 (15 μM)
10 μl AMV-RT (5 units/μl)

Forty-five (45 μl) of the RT-PCR master mix was dispensed into MicroAmp-brand strip-well tubes on ice. Five (5) μl of each reaction was then added to the master mix. The reactions were placed in the PE 9600 thermocycler and cycled as follows:

| | | |
|---|---|---|
| 48° C. | 45 minutes | 1 cycle |
| 96° C. | 2 minutes | 1 cycle |
| 94° C. | 15 seconds | |
| 65° C. -> 55° C. | 1 minute | 12 cycles |
| 72° C. | 1.5 minutes | |
| 72° C. | 5 minutes | 1 cycle |
| 4° C. | Soak | |

Twenty (20) μl of each RT-PCR reaction was then loaded onto a 1% TBE agarose gel with ethidium bromide staining and run for 1 hour at 80V.

Results:

The results are shown in FIGS. 2, 3, and 4.

FIGS. 2 (rat) and 3 (human)—Lane Nos:

1. 200 bp DNA Step Ladder

2. No template Control

3. No Lysate/no RNasin—Full product

4. No Lysate—Full product

5. No Lysate—Full product

6. No Lysate—Full product

7. +Lysate/no RNasin

8. +Lysate/no RNasin

9. +Lysate/no RNasin

10. +Lysate+RNasin

11. +Lysate+RNasin

12. +Lysate+RNasin

Quantitation of the band intensities in FIG. 2 and FIG. 3 was performed using densitometry and the ratio of luciferase product (upper band 1.6 Kb) to kanamycin product (lower band 1.2 Kb) were determined. The ratios were then averaged over n=3:

| | top | bottom | ratio | Average | SD |
|---|---|---|---|---|---|
| 2 (+ control) | 213875 | 429975 | 0.4974 | 0.5696 | 0.0867 |
| 3 (+ control) | 207031 | 394050 | 0.5253 | | |
| 4 (+ control) | 208742 | 301035 | 0.6934 | | |
| 5 (+ control) | 205821 | 365820 | 0.5626 | | |
| 6 (lysate) | 223445 | 302907 | 0.7377 | 0.7467 | 0.0584 |
| 7 (lysate) | 228114 | 267729 | 0.8520 | | |
| 8 (lysate) | 236778 | 305877 | 0.7740 | | |
| 9 (RNasin) | 239481 | 368160 | 0.6504 | 0.6036 | 0.0701 |
| 10 (RNasin) | 242121 | 462849 | 0.5231 | | |
| 11 (RNasin) | 245322 | 384800 | 0.6375 | | |

| | AVG | SD |
|---|---|---|
| Control | 0.5696 | 0.0867 |
| Lysate-treated | 0.7467 | 0.0584 |
| Lysate + RNasin | 0.6036 | 0.0701 |

A two-tailed t-test was then performed assuming unequal variances. The results were as follows:
Lysate/Control

t-Test: Two-Sample Assuming Unequal Variances:

| | Lysate | Control |
|---|---|---|
| Mean | 0.7879 | 0.569675 |
| Variance | 0.00341103 | 0.007516916 |
| Observations | 3 | 4 |
| Hypothesized Mean Difference | 0 | |
| Df | 5 | |
| t Stat | 3.973485473 | |
| P(T <= t) one-tail | 0.005299643 | |
| t Critical one-tail | 2.015049176 | |
| P(T <= t) two-tail | 0.010599285 | |
| t Critical two-tail | 2.570577635 | |

Lysate/RNasin

t-Test: Two-Sample Assuming Unequal Variances:

| | Lysate | RNasin |
|---|---|---|
| Mean | 0.7879 | 0.603666667 |
| Variance | 0.00341103 | 0.004909843 |
| Observations | 3 | 3 |
| Hypothesized Mean Difference | 0 | |
| Df | 4 | |
| t Stat | 3.498197957 | |
| P(T <= t) one-tail | 0.012468416 | |
| t Critical one-tail | 2.131846486 | |
| P(T <= t) two-tail | 0.024936833 | |
| t Critical two-tail | 2.776450856 | |

Control/RNasin

t-Test: Two-Sample Assuming Unequal Variances:

| | RNasin | Control |
|---|---|---|
| Mean | 0.603666667 | 0.569675 |
| Variance | 0.004909843 | 0.007516916 |
| Observations | 3 | 4 |
| Hypothesized Mean Difference | 0 | |
| Df | 5 | |
| t Stat | 0.573267997 | |
| P(T <= t) one-tail | 0.295640794 | |
| t Critical one-tail | 2.015049176 | |
| P(T <= t) two-tail | 0.591281587 | |
| t Critical two-tail | 2.570577635 | |

For lysate-treated and Full product control, p<0.05, a significant difference.

For lysates-treated and RNasin-protected, p<0.05, a significant difference.

For control Full-product and RNasin-protected, p>0.05, an insignificant difference.

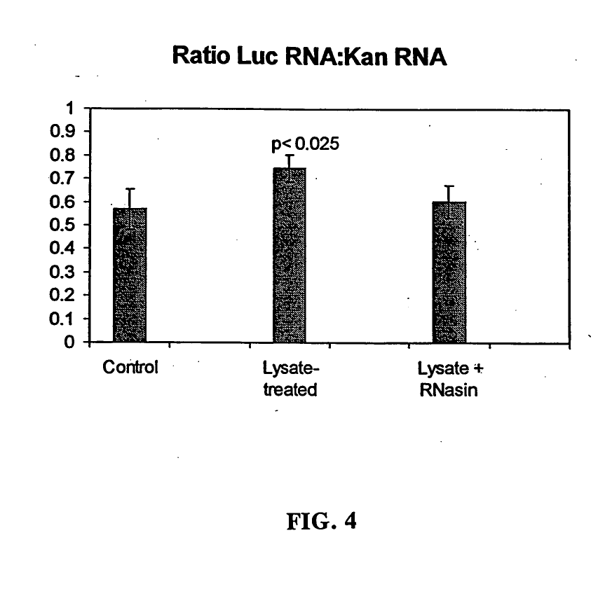

These results are presented graphically in FIG. 4.

This Example shows that there is a significant difference between the lysate-treated samples and the control samples and between the lysate-treated samples and the RNasin-treated samples. There is no significant difference between the control samples and the RNasin-treated samples. In short, there is no difference in the yield of RT-PCR product obtained in the reactions where the inhibitor is added to lysate, but there is a significant difference in the yield of product when no inhibitor is added to the lysate.

### Example 3

### Effect of Heating RNase in Presence of RNase Inhibitor

The Example illustrates the effect of heating the RNase in the presence of RNase inhibitor. The experiment was conducted as follows: Agar was mixed with RNA and a pH indicator. RNase degradation of RNA releases the nucleotides, thereby decreasing the local pH. This turns the pH indicator pink. The agar was poured in a petri dish and allowed to solidify.

Three solutions were assembled in duplicate in 0.5 ml microfuge tubes. The compositions were as follows:

| Component | RNAse Alone | RNAse + HR | RNAse + RR |
|---|---|---|---|
| Water | 80 μl | 70 μl | 70 μl |
| RNAse A | 20 μl | 20 μl | 20 μl |
| (0.1 mg/ml)* | | | |
| Human RNAsin | 0 μl | 10 μl | 0 μl |
| (40 U/μl) | | | |
| Rat RNAsin | 0 μl | 0 μl | 10 μl |
| (40 U/μl | | | |

*RNAse A was prepared in a buffer containing Ribonuclease A (Sigma R4875) in water.

One of the duplicate solutions was heated at 70° C. for 5 min, and then allowed to cool to room temperature. The other of the duplicate solutions was kept at room temperature the entire time.

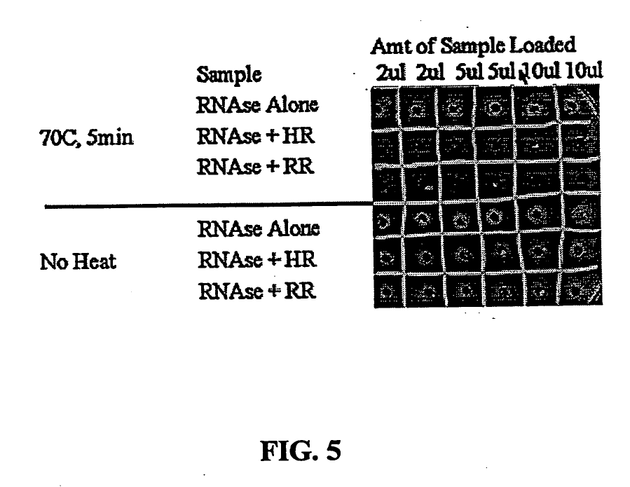

The dish was gridded and wells were cored into the gel for loading the different samples. Samples of these solutions were then placed in the wells cored into the agar plate. As shown in FIG. 5, the top half of the plate comprises samples which were heated, while the bottom half comprises samples which were not heated. The heated samples were, from top to bottom, RNase alone; RNase plus human RNase inhibitor; and RNase plus recombinant RNase inhibitor (rat-derived). The non-heated samples are in the same order. From left to right, the lanes show the samples were added in volumes of 2 μl, 2 μl, 5 μl, 5 μl, 10 μl, and 10 μl, respectively.

The results of the experiment show that for both the heated and unheated rows containing the RNase alone, there is a dark halo indicating degradation of RNA. For the rows containing RNase and human RNase inhibitor and rat-derived RNase inhibitor, there is no halo, indicating that there is no degradation of RNA. For the rows containing RNase and human RNase inhibitor or rat-derived RNase inhibitor that were not heated-treated, there is a weak halo around all the cores, indicating that even for non-heat-treated samples, the protection of RNA by RNase inhibitor is not complete. In contrast, there is complete inhibition for the heat-treated samples even at high volumes of added RNase.

### Example 4

### RNA Degradation By RNase in Presence of Inhibitor, Heated & Non-Heated

This Example was performed to examine the breakdown of RNA by RNase in the presence of RNase inhibitor and buffer with and without heating. The experiment was performed by preparing two identical agar plates in which the agar was mixed with RNA and a pH indicator.

Five solutions were assembled in duplicate in 0.55 ml microfuge tubes. The compositions were:

| Compo- | RNAse | RNAse + | RNAse + | RNAse + | No RNAse |
|---|---|---|---|---|---|
| nent | Alone | RNAsin | SB* | Buffer B** | (control) |
| Water | 80 μl | 70 μl | 70 μl | 70 μl | 100 μl |
| RNAse | 20 μl | 20 μl | 20 μl | 20 μl | 0 μl |
| A* | | | | | |
| RNAsin | 0 μl | 10 μl | 0 μl | 0 μl | 0 μl |
| Plus | | | | | |
| (40 U/μl) | | | | | |
| Storage | 0 μl | 0 μl | 10 μl | 0 μl | 0 μl |
| Buffer** | | | | | |
| Buffer | 0 μl | 0 μl | 0 μl | 10 μl | 0 μl |
| B*** | | | | | |

*RNAse A = RNAse A was prepared in a buffer containing Ribonuclease A (Sigma R4875) in water.
**Storage Buffer = 20 mM HEPES-KOH (pH 7.6 at 4° C.), 50 mM KCl, 8 mM DTT, 50% (v/v) glycerol
***Buffer B = 60 mM Tris-Cl, pH 7.9 (at 37° C.), 60 mM MgCl₂, 500 mM NaCl, 10 mM DTT

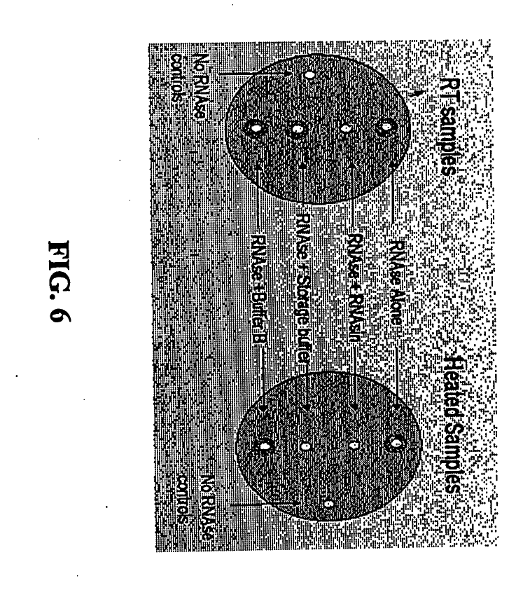

One tube of each duplicate was heated at 70° C. for 5 min, and then allowed to cool to room temperature. The other tube of each duplicate was kept at room temperature the entire time. Samples, 10 μl each, of these solutions were then placed into the wells in the agar plates. The results of this experiment, as illustrated in the schematic in FIG. 6.

The plates were loaded identically, with the exception that the plate on the left was loaded with samples incubated at room temperature, while the plate on the right was loaded with samples that were heated to 70° C. The plates were loaded, top to bottom: RNase alone, RNase+“RNasin” RNase inhibitor in Promega Storage Buffer; RNase+storage buffer; RNase+Promega Buffer B. The results of the experiment, shown in FIG. 6, indicate that, for the unheated samples, inhibition of RNase occurs in the presence of the inhibitor only. For the heated samples, inhibition occurs only in the presence of the inhibitor and the storage buffer. These results indicate that for protection of RNA at both room temperature and increased temperatures, RNase and buffer must be added while the mix is being prepared at room temperature.

### Example 5

### Inhibition of Wheat Germ RNases with Rat RNasin

The purpose of this Example is to determine whether pre-heated rat RNasin is an effective inhibitor of the RNases present in wheat germ extract.

Material:

Wheat Germ Extract (Promega Pt#L481A, Lt#12204104)

RNasin Plus: 40 units/μl (Promega Pt#N261, Lt#165682)

Luciferase mRNA: 1 mg/ml (Promega Pt#L456A, Lt #14937403)

AcessQuick™RT-PCR System (Promega Pt#A1 703, Lt#158304)

Experimental:

The following reactions were assembled without addition of luciferase mRNA:

| | | | Wheat Germ | |
|---|---|---|---|---|
| reaction # | Luc mRNA | Nanopure | Extract | Rat RNasin |
| 1 | — | 30 μl | — | — |
| 2 | 1 μl (1 μg) | 29 μl | — | — |
| 3 | 1 μl (1 μg) | 9 μl | — | 20 μl |
| 4 | 1 μl (1 μg) | — | 1 μl | 20 μl |
| 5 | 1 μl (1 μg) | — | 1 μl | 20 μl |
| 6 | 1 μl (1 μg) | 9 μl | 1 μl | 10 μl |
| 7 | 1 μl (1 μg) | 14 μl | 1 μl | 5 μl |

Reaction Nos. 1 through 4 were kept at room temperature. Reaction Nos. 5 through 7 were heated at 70° C. for 15 minutes and then allowed to cool to room temperature.

One (1) μl (1 μg) of luciferase mRNA was then added to the reactions as indicated.

The reactions were then incubated at 37° C. for 60 minutes.

An RT-PCR master mix was assembled on ice as follows, using components available from Promega Corp.:
250 μl Access Quick 2× Master Mix
220 μl Nuclease Free Water
10 μl Luciferase Upstream primer, Promega Pt. #20247 (15 82 M)
10 μl Luciferase Downstream primer, Promega Pt. #20818 (15 μM)
10 μl AMV-RT (5 units/μl)

Forty-five (45) μl of the RT-PCR master mix was dispensed into “MicroAmp”-brand strip-well tubes on ice. Five (5) μl of each reaction was then added to the master mix. The reactions were placed in the PE 9600 thermocycler and cycled as follows:

| | | |
|---|---|---|
| 48° C. | 45 minutes | 1 cycle |
| 96° C. | 2 minutes | 1 cycle |
| 94° C. | 15 seconds | |
| 65° C. -> 55° C. | 1 minute | 20 cycles |
| 72° C. | 1.5 minutes | |
| 72° C. | 5 minutes | 1 cycle |
| 4° C. | Soak | |

Fifteen (15) μl of each RT-PCR reaction was then loaded onto a 1% TBE agarose gel with ethidium bromide staining and run for 1 hour at 80V.

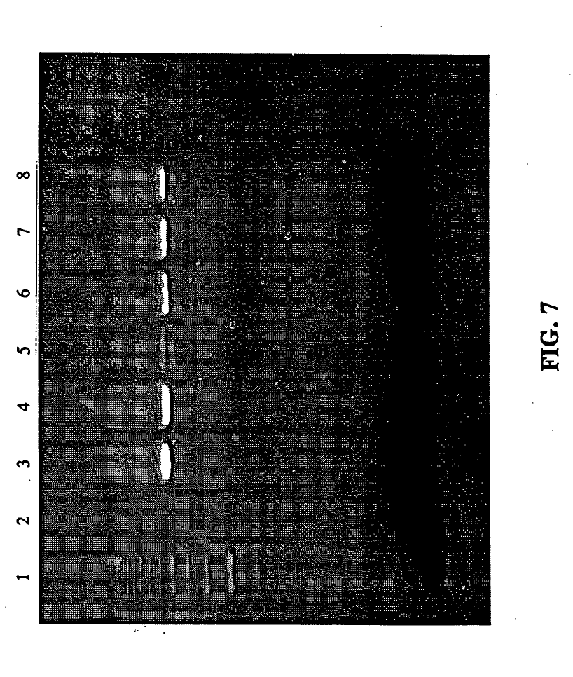

The results are shown in FIG. 7 (WGE=wheat germ extract):
Lane #1—200 b.p. DNA Step Ladder
2—No template
3—Full Product
4—RNasin Only/No WGE
5—WGE+RNasin @RT
6—WGE+Rnasin@70° C. (20 μl)
7—WGE+Rnasin@70° C. (10 μl)
8—WGE+RNasin@70° C. (5 μl)

This Example demonstrates that heat-treated rat RNasin is inhibiting some of the RNases present in the wheat germ extract, although the inhibition is not complete. See lane 5 of FIG. 7 and compare to lanes 6, 7, and 8.

### Example 6

### More Inhibition of Wheat Germ RNases with Rat RNasin:

The purpose of this Example, like that of Example 4, was to determine whether pre-heated rat RNasin is an effective inhibitor of the RNases present in wheat germ extract. Slightly different buffers were used in this Example, including a buffer with and without added DTT (to assess the effects of DTT on the reactions).

Materials:

Wheat Germ Extract (Promega Pt#L481A Lt#12204104)

RNasin Plus: 40 units/μl (Promega Pt#N261 Lt#165682)

Luciferase mRNA: 1 mg/ml (Promega Pt#L456A Lt #14937403)

AccessQuick™RT-PCR System (Promega Pt#A1703 Lt#158304)

RNasin Storage Buffer (Promega Pt #BN251 Lt#147681)

RNasin Storage Buffer plus DTT:
20 mM HEPES-KOH, pH 7.6
50 mM KCl
8 mM DTT
50% glycerol

Experimental:

The following reactions were assembled without addition of luciferase mRNA:

| | | | Wheat | | Storage | |
|---|---|---|---|---|---|---|
| Reaction | Luc | | Germ | Rat | buffer + | Storage [symbol] |
| # | mRNA | Nanopure | Extract | RNasin | DTT | DT [symbol] |
| 1 | — | 30 μl | — | — | — | — |
| 2 | 1 μl (1 μg) | 29 μl | — | — | — | — |
| 3 | 1 μl (1 μg) | 9 μl | — | 20 μl | — | — |
| 4 | 1 μl (1 μg) | 28 μl | 1 μl | — | — | — |
| 5 | 1 μl (1 μg) | 28 μl | 1 μl | — | — | — |
| 6 | 1 μl (1 μg) | 8 μl | 1 μl | 20 μl | — | — |
| 7 | 1 μl (1 μg) | 8 μl | 1 μl | 20 μl | — | — |
| 8 | 1 μl (1 μg) | 18 μl | 1 μl | 10 μl | — | — |
| 9 | 1 μl (1 μg) | 23 μl | 1 μl | 5 μl | — | — |
| 10 | 1 μl (1 μg) | 8 μl | 1 μl | — | 20 μl | — |
| 11 | 1 μl (1 μg) | 8 μl | 1 μl | — | 20 μl | — |
| 12 | 1 μl (1 μg) | 8 μl | 1 μl | — | — | 20 [symbol] |
| 13 | 1 μl (1 μg) | 8 μl | 1 μl | — | — | 20 [symbol] |

Reaction Nos. 1 through 4, 6, 10, and 12 were kept at room temperature. Reaction Nos. 5, 7, 8, 9, 11, 13, and 15 were heated at 70° C. for 15 minutes and then allowed to cool to room temperature.

One (1) μl (1 μg) of luciferase mRNA was then added to the reactions as indicated.

The reactions were then incubated at 37° C. for 60 minutes.

An RT-PCR master mix was assembled on ice as follows:
250 μl Access Quick 2× Master Mix
220 μl Nuclease Free Water
10 μl Luciferase Upstream primer, Promega Pt. #20247 (15 μM)
10 μl Luciferase Downstream primer Promega Pt. #20818 (15 μM)
10 μl AMV-RT (5 units/μl)

Forty-five (45) μl of the RT-PCR master mix was dispensed into “MicroAmp”-brand strip-well tubes on ice. Five (5) μl of each reaction was then added to the master mix. The reactions were placed in the PE 9600 thermocycler and cycled as follows:

| | | |
|---|---|---|
| 48° C. | 45 minutes | 1 cycle |
| 96° C. | 2 minutes | 1 cycle |
| 94° C. | 15 seconds | |
| 65° C. -> 55° C. | 1 minute | 20 cycles |
| 72° C. | 1.5 minutes | |
| 72° C. | 5 minutes | 1 cycle |
| 4° C. | Soak | |

Fifteen (15) μl of each RT-PCR reaction was then loaded onto a 1% TBE agarose gel with ethidium bromide staining and run for 1 hour at 80V.

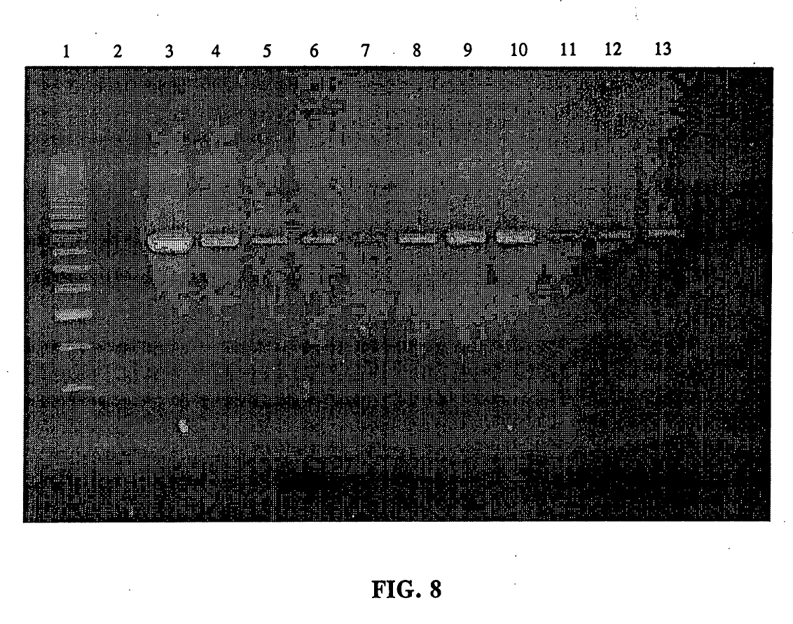

The results are shown in FIG. 8.

Lane #1—200 b.p. DNA Step Ladder
2—No template
3—Full Product
4—RNasin Only/No WGE
5—WGE RT Only/No RNasin
6—WGE 70° C. Only/No RNasin
7—WGE RT+RNasin RT
8—WGE 70° C.+RNasin 70° C. (20 μl)
9—WGE 70° C.+RNasin 70° C. (10 μl)
10—WGE 70° C.+RNasin 70° C. (5 μl)
11—WGE RT+Storage Buffer w/DTT RT
12—WGE 70° C.+Storage Buffer w/DTT 70° C.
13—WGE 70° C.+Storage Buffer no DTT 70° C.
14—WGE RT+Storage Buffer w/DTT RT

NB: Reaction Nos. 12 and 13, in lanes 13 and 14, were accidentally inverted upon loading the gel.

As in Example 5, this Example shows that the present invention is capable of inhibiting the wheat germ extract RNases, but not completely. Specifically, compare the amount of product obtained in lane 7 vs. lanes 8 through 10. Also, an interesting observation from lanes 11 through 14: Storage Buffer with or without DTT is capable of providing some protection as long as it is heated. It appears as if all factors contribute in some fashion to the synergistic inhibitory effect seen by the combination of rat RNasin, Storage Buffer, DTT, and heat.

It is understood that the invention is not confined to the particular construction and arrangement of parts herein illustrated and described, but embraces such modified forms thereof as come within the scope of the following claims.

1. A method for protecting RNA from enzymatic degradation by RNases, the method comprising:
(a) to a first solution containing RNA or to which RNA will subsequently be added, adding a second solution, the second solution comprising an amount of an RNase inhibitor protein disposed in a buffer comprising an amount of DTT, to yield a mixture, wherein the amount of RNase inhibitor protein in the second solution is sufficient to protect RNA from enzymatic degradation by RNases, and wherein the amount of DTT in the second solution is sufficient to make the mixture comprise at least about 50 μM DTT; and then
(b) heating the mixture of step (a) to a temperature no less than about 70° C. for a time sufficient to inhibit RNase activity present in the mixture; whereby RNA present in the mixture or subsequently added to the mixture is protected from enzymatic degradation by RNases.

2. The method of claim 1, wherein in step (a), the amount of DTT in the second solution is sufficient to make the mixture comprise at least about 100 μM DTT.

3. The method of claim 1, wherein in step (a), the amount of DTT in the second solution is sufficient to make the mixture comprise at least about 1.0 mM DTT.

4. The method of claim 1, wherein in step (a), the RNase inhibitor protein is derived from porcine, rat, human placental, or recombinant human placental sources.

5. The method of claim 1, wherein in step (b), the mixture does not contain RNA and further wherein the mixture is heated to a temperature no less than about 90° C.

6. The method of claim 1, wherein in step (b), the mixture is heated for at least about two (2) minutes.

7. The method of claim 1, wherein in step (b), the mixture is heated for at least about five (5) minutes.

8. The method of claim 1, which is a method of protecting RNA from enzymatic degradation by RNase A, RNase B, RNase C, and RNase 1.

9. A method of inactivating RNases in a first solution containing RNA and suspected of containing RNases, the method comprising:
(a) to the first solution, adding a second solution comprising an RNase inhibitor protein deposited in a buffer comprising an amount of DTT to yield a mixture, wherein the amount of DTT in the second solution is sufficient to make the mixture comprise at least about 50 μM DTT; and then
(b) heating the mixture of step (a) to a temperature of at least about 70° C. for a time sufficient to inhibit RNase activity present in the mixture; whereby RNases present in the first solution, if any, are inactivated.

10. The method of claim 9, wherein in step (a), the amount of DTT in the second solution is sufficient to make the mixture comprise at least about 100 μM DTT.

11. The method of claim 9, wherein in step (a), the amount of DTT in the second solution is sufficient to make the mixture comprise at least about 1.0 mM DTT.

12. The method of claim 9, wherein in step (a), the RNase inhibitor protein is derived from porcine, rat, human placental or recombinant human placental sources.

13. The method of claim 9, wherein in step (b), the mixture is heated for at least about two (2) minutes.

14. The method of claim 9, wherein in step (b), the mixture is heated for at least about five (5) minutes.

15. The method of claim 9, which is a method of inactivating any RNase A, RNase B, RNase C, and RNase 1 present in the first solution.

16. A method of storing RNA under conditions that protect the RNA from enzymatic degradation by RNases, the method comprising:
(a) to a first solution containing isolated RNA or to which isolated RNA will subsequently be added, adding a second solution comprising an RNase inhibitor protein in a buffer comprising an amount of DTT, to yield a mixture, wherein the amount of DTT in the second solution is sufficient to make the mixture comprise at least about 50 μM DTT; and then
(b) heating the mixture of step (a) to a temperature of at least about 70° C. for a time sufficient to inhibit RNase activity present in the mixture; and then
(c) cooling the mixture and storing it in an air-tight container.

17. The method of claim 16, wherein in step (a), the amount of DTT in the second solution is sufficient to make the mixture comprise at least about 100 μM DTT.

18. The method of claim 16, wherein in step (a), the amount of DTT in the second solution is sufficient to make the mixture comprise at least about 1.0 mM DTT.

19. The method of claim 16, wherein in step (a), the RNase inhibitor protein is derived from porcine, rat, human placental, or recombinant human placental sources.

20. The method of claim 16, wherein in step (b), the mixture does not contain RNA and further wherein the mixture is heated to a temperature no less than about 90° C.

21. The method of claim 16, wherein in step (b), the mixture is heated for at least about two (2) minutes.

22. The method of claim 16, wherein in step (b), the mixture is heated for at least about five (5) minutes.

23. A method of performing RT-PCR and quantitative RT-PCR, the method comprising:
(a) prior to undergoing thermal cycling, adding to an RT-PCR reaction cocktail containing RNA or to which RNA will subsequently be added, an amount of a solution comprising an RNase inhibitor protein in a buffer comprising an amount of DTT, to yield a mixture, wherein the amount of the solution added is sufficient to protect any RNA present in the RT-PCR reaction cocktail from enzymatic degradation during a first round of thermocycling, and wherein the amount of DTT in the solution is sufficient to make the mixture comprise at least about 50 μM DTT; and then
(b) adding RNA template to the mixture of step (a) if RNA is absent, and then conducting an RT-PCR reaction on the mixture of step (a), whereby RNA in the mixture is protected from enzymatic degradation by RNases present in the RT-PCR reaction cocktail and is further protected from enzymatic degradation by RNases during the first round of thermocycling and throughout the RT-PCR reaction.

24. The method of claim 23, wherein in step (a), the amount of DTT in the solution is sufficient to make the mixture comprise at least about 100 μM DTT.

25. The method of claim 23, wherein in step (a), the amount of DTT in the solution is sufficient to make the mixture comprise at least about 1.0 mM DTT.

26. The method of claim 23, wherein in step (a), the RNase inhibitor protein is derived from porcine, rat, human placental, or recombinant human placental sources.

27. The method of claim 23, wherein after step (a) and prior to step (b), the mixture is heated to at least about 70° C.

28. The method of claim 23, wherein in step (a) the RT-PCR reaction cocktail does not contain RNA; and after step (a) and prior to step (b), the mixture is heated to at least about 90° C.

29. A method of performing RT-PCR and quantitative RT-PCR, the method comprising:
(a) to an RT-PCR reagent mixture, adding a first solution containing an RNase inhibitor protein in a buffer, the buffer comprising an amount of DTT, to yield a second solution, wherein the amount of DTT in the buffer is sufficient to make the second solution comprise at least about 50 μM DTT; and
(b) heating the second solution to at least about 70° C. for a time sufficient to inhibit RNase activity present in the second solution; and then
(c) adding RNA to the second solution to yield an RNA mixture; and then
(d) conducting an RT-PCR reaction on the RNA mixture of step (c); whereby the RNA in the RNA mixture is protected from enzymatic degradation by RNases present in the second solution and whereby the RNA in the mixture is further protected from RNases during the RT-PCR reaction.

30. The method of claim 29, wherein in step (a), the amount of DTT in the buffer is sufficient to make the second solution comprise at least about 100 μM DTT.

31. The method of claim 29, wherein in step (a), the amount of DTT in the buffer is sufficient to make the second solution comprise at least about 1.0 mM DTT.

32. The method of claim 29, wherein in step (a), the RNase inhibitor protein is derived from porcine, rat, human placental, or recombinant human placental sources.

33. The method of claim 29, wherein in step (b), the mixture is heated to at least about 90° C.

34. The method of claim 29, wherein in step (b), the mixture is heated for at least about two (2) minutes.

35. The method of claim 29, wherein in step (b), the mixture is heated for at least about five (5) minutes.

36. A method of inactivating a prokaryotic or plant RNase comprising:
(a) to a first solution suspected of containing a prokaryotic or plant RNase, adding a second solution comprising an RNase inhibitor protein in a buffer comprising an amount of DTT, to yield a mixture, wherein the amount of DTT in the buffer is sufficient to make the mixture comprise at least about 50 μM DTT; and then
(b) heating the mixture of step (a) to a temperature of at least about 70° C. for a time sufficient to inhibit prokaryotic or plant RNase activity present in the mixture, whereby prokaryotic and plant RNase present in the first solution is inactivated.

37. The method of claim 36, wherein in step (a), the amount of DTT in the buffer is sufficient to make the mixture comprise at least about 100 μM DTT.

38. The method of claim 36, wherein in step (a), the amount of DTT in the buffer is sufficient to make the mixture comprise at least about 1.0 mM DTT.

39. The method of claim 36, wherein in step (a), the RNase inhibitor protein is derived from porcine, rat, human placental, or recombinant human placental sources.

40. The method of claim 36, wherein in step (b), the mixture is heated to at least about 90° C.

41. The method of claim 36, wherein in step (b), the mixture is heated for at least about two (2) minutes.

42. The method of claim 36, wherein in step (b), the mixture is heated for at least about five (5) minutes.

43. The method of claim 36, wherein in step (a), the first solution is suspected of containing E. coli RNase; and in step (b), the mixture is heated for a time sufficient to inhibit E. coli RNase activity present in the mixture.


SEQUENCE LISTING (6 sequences)

SEQ ID NO: 1 - DNA, 13 bp, Artificial
  Luciferase RT-PCR reverse primer
cgccccctcggag

SEQ ID NO: 2 - DNA, 11 bp, Artificial
  Luciferase RT-PCR forward primer
gaaaggcccgg

SEQ ID NO: 3 - DNA, 20 bp, Artificial
  Kan RT-PCR reverse primer
gggatcctctagagtcgcca

SEQ ID NO: 4 - DNA, 20 bp, Artificial
  Kan RT-PCR forward primer
ttgggcgtgtctcaaaatct

SEQ ID NO: 5 - DNA, 13 bp, Artificial
  Luciferase RT-PCR reverse primer
cgccccctcggag

SEQ ID NO: 6 - DNA, 11 bp, Artificial
  Luciferase RT-PCR forward primer
gaaaggcccgg